(19) United States
(12) Patent Application Publication
(10) Pub. No.: US 2022/0127571 A1
(43) Pub. Date: Apr. 28, 2022

(54) CELL POPULATIONS WITH IMPROVED PRODUCTION AND THERAPEUTIC CHARACTERISTICS

(71) Applicant: GPB SCIENTIFIC, INC., Richmond, PA (US)

(72) Inventors: Anthony WARD, Rancho Santa Fe, CA (US); Alison SKELLEY, Riverside, CA (US); Khushroo GANDHI, Palo Alto, CA (US); Roberto CAMPOS-GONZALEZ, Carlsbad, CA (US)

(73) Assignee: GPB SCIENTIFIC, INC., Richmond, VA (US)

(21) Appl. No.: 17/425,935

(22) PCT Filed: Jan. 28, 2020
(86) PCT No.: PCT/US2020/015377

§ 371 Date: Jul. 26, 2021

Related U.S. Application Data
(60) Provisional application No. 62/798,469, filed on Jan. 29, 2019.
(60) Provisional application No. 62/814,285, filed on Mar. 5, 2019.

(51) Int. Cl. C12N 5/0783; C12N 5/00; C12N 15/85; A61K 35/17

(52) U.S. Cl. CPC C12N 5/0636; C12N 5/0087; C12N 2501/998; A61K 35/17; C12N 15/85

(57) ABSTRACT

The present invention is directed to improved methods of preparing cells and compositions for therapeutic uses.

### CROSS REFERENCE TO RELATED APPLICATIONS

The present application claims the benefit of U.S. provisional patent application No. 62/798,469, filed on Jan. 29, 2019 and the benefit of U.S. provisional patent application No. 62/814,285, filed on Mar. 5, 2019.

### FIELD OF THE INVENTION

The present invention is in the field of developing cells for therapeutic uses and more specifically to improving the yield of the most pharmaceutically desirable cells by controlling the rate at which they divide and differentiate during processing.

### BACKGROUND OF THE INVENTION

Cells that are used therapeutically often develop in stages that are induced either in vivo or in vitro. For example, in response to antigens or stimulation with anti-CD3 and anti-CD28 antibody, naive T cells begin a process in which they develop into T memory stem cells, followed by central memory T cells, effector memory cells and finally short lived effector T cells (see Gattinoni, et al.; Blood 121(4):567-568 (2013)). Factors known to be capable of affecting this process include IL-7, IL-15 and TWS119 (promoting the progression of naive T cells to T memory stem cells) and IL-2 (promoting the development of naive T cells into effector memory cells (Id.)

Apart from natural processes, the collection of blood initiates a cascade of events that is well documented. In addition to the classical coagulation cascade following intrinsic or extrinsic platelet activation, changes in hemodynamic balance (relative hematocrit, plasma concentration, anticoagulant type and presence etc.) also initiate cellular responses in an attempt to restore hemostasis. More generally, excess contact, perturbation and resultant cell signaling has been documented to induce cell activation, anergy and even tonic signaling (increased frequency of T cell:B Cell interactions) within a sample. Thus, cells collected for therapy are changed by the act of collection and processing in ways that affect their ability to respond therapeutically.

Cells often must also be genetically engineered to realize their full potential as therapeutic agents. In such cases, the cells will typically need to divide to successfully integrate a genetic insert that provides them with therapeutically valuable attributes. For example, CAR-T cells must be engineered to allow them to effectively target tumor cells.

The type of T cell that is preferred for the making of CAR T cells is a relatively undifferentiated cell, such as T memory cell and or, more preferably, a T memory stem cell. Obtaining a high yield of these cells will depend on both eliminating factors that may be present in cell preparations that steer cells to unwanted ends and adding factors that steer the cells to their most therapeutically desirable state. It should also be recognized that, as the number of T cell doublings increases, the proportion of less desirable cells increases. Therefore, controlling the number of doublings is important.

### SUMMARY OF THE INVENTION

The present invention is based on the concept that the yield of genetically engineered cells with therapeutically valuable phenotypes can be improved by taking steps to control the extent to which the cells become activated, divide and differentiate during processing.

The invention is directed to a method of producing a population of genetically engineered target cells from target cells that are not terminally differentiated. In general, the target cells should be therapeutically relevant (i.e., cells that have a therapeutic use) nucleated cells greater than 3.5 μm in diameter. They may be: a) leukocytes, including neutrophils, basophils, eosinophils, lymphocytes (including B cells, T cells and natural killer cells), monocytes, macrophages, mast cells, dendritic cells; b) stem cells, including i) stem cells that develop into leukocytes such as stem cells with CD34 and/or CD38 markers and leukocyte lineage negative cells, and ii) stem cells that develop into cells other than leukocytes; and c) erythroid precursor cells. Especially preferred target cells include naïve T cells and T memory stem cells.

The first step in the present method, step a), comprises obtaining a sample of target cells. The sample may either be obtained from a patient directly by the party performing other steps in the process or it may be provided by someone else. For example, an apheresis sample may be obtained by the party carrying out the separation of cells from a healthcare provider who collected the sample or the party performing the separation may obtain the sample from a patient directly. For the purposes herein, being given a sample by another party that did the collection will constitute “obtaining” the sample. The samples will typically also contain “contaminants” which, in the context of the present invention, are cells, proteins or other factors that promote the proliferation or differentiation of the target cells. The contaminants may be substances secreted by cells of the immune system (e.g., T cells) that have an effect on other cells or may be platelets or factors released by platelets. Cellular contaminants, and especially platelets, will typically be found in samples of blood or products derived from the processing of blood such as apheresis or leukapheresis samples.

In step b) of the method, the target cells in the sample are separated from the contaminants using a size and/or affinity based separation method to obtain an enriched population of target cells. The separation should be carried out soon after the cells are collected and reduce the contaminants by at least 70% compared to the sample before separation and/or reduce the ratio of contaminants to target cells by at least 70% (with reductions of 80%, 85%, 90%, or 95% being preferred). In order to avoid activation, cells should preferably not be centrifuged as part of sample collection and processing or, if centrifugation was used during collection, e.g., as part of an apheresis or leukapheresis procedure, it should not be used thereafter, i.e., as part of the separation in step b) or, as described below, as part of the genetic engineering of step c). In addition, factors that promote proliferation, promote the differentiation of the target cells to a more fully differentiated state or otherwise redirect the proliferation or differentiation of cells should not be added prior to separation.

The most preferred method for separating the target cells is a size-based method performed using a microfluidic device. The device will generally have an array of obstacles arranged in rows, with each subsequent row of obstacles being shifted laterally with respect to a previous row, and positioned so as to differentially deflect cells of a predetermined size (including the target cells) to a first outlet where they are recovered as a product, and to direct cells or particles of less than the predetermined size to a second outlet where they may be collected or discarded as waste. A preferred method of separation is by deterministic lateral displacement (DLD). As discussed further herein, DLD procedures and microfluidic devices for performing DLD are well known in the art.

In order to minimize the effect of the contaminants on target cells, the separation step should generally be initiated within 5 hours after the sample comprising target cells is collected or otherwise obtained. More preferably, this should occur within 3 hours, 2 hours or one hour. Optionally, one or more agents may added to the sample of target cells at the time sample is collected or during processing, to reversibly inhibit proliferation and/or differentiation. These agents would generally need to be removed or reversed sufficiently to allow cells to divide at the time, or shortly prior to the time, that they are being genetically engineered.

Once the separation step is completed, the target cells in the enriched population are, in step c), genetically engineered to produce target cells with a therapeutically useful phenotype. In an especially preferred embodiment, the target cells are T cells that are engineered to produce chimeric antigen receptors (CARs) on their surface. The genetic engineering of cells may be delayed (for example if preparations are frozen) but generally, this should occur within 1 week after the sample comprising target cells is collected or otherwise obtained. In some embodiments, shorter periods (3 days, 1 day, 6 hours, or 3 hours) would be preferable.

Although excessive proliferation and differentiation of cells is to be avoided, cells typically must undergo division in order for recombinantly introduced nucleotide sequences to become integrated into the cell's genome. Thus, it may be desirable to add one or more factors that promote the proliferation of target cells before and/or during genetic engineering. However, these factors should generally not be added to cells more than about three days (and preferably no more than two days, one day, five hours, three hours or one hour) before genetic engineering is initiated. For the purposes herein, genetic engineering is considered to be initiated when cells are first combined with the recombinant nucleic acids that will be transferred into the cells. Similarly, for the purposes herein, genetic engineering is considered to be completed when the transfer of recombinant nucleic acids into cells is no longer occurring, e.g., because the recombination process has been terminated or factors needed for the process have been removed. Factors that promote the proliferation of target cells may include agents released by immune cells, cytokines, peptides, peptide receptor complexes, and antibodies used either alone or in conjunction with costimulatory molecules. After genetic engineering, a further separation may be performed to remove reagents and stimulatory factors. This should typically be done within one or two days after genetic engineering is completed. Preferably, the reagents and any other stimulatory factors are removed using a size based separation procedure such as DLD. The cells may then optionally be cultured to expand their number and finally collected for use therapeutically, usually as part of a pharmaceutical composition.

A primary objective is to minimize proliferation and differentiation of target cells and thereby increase the number of relatively undifferentiated cells undergoing genetic engineering and that will eventually be available for use therapeutically. In this regard, at least 70% (and more preferably 80%, 90% or 95%) of the target cells should preferably not have divided more than once from the time that they are collected or otherwise obtained until the separation of step b) is initiated. Similarly at least 70% (and preferably 80%, 90% or 95%) of the target cells should preferably not have not been activated or divided more than once at the time that the genetic engineering of step c) is initiated, i.e., at the time that cells are first combined with the recombinant nucleic acids being transferred into the cells. Similarly, it is preferred that 70% (and preferably 80%, 90% or 95%) of the target cells not have divided more than twice (and more preferably not more than once) at the time that the genetic engineering of step c) is complete and/or that the population of cells not have undergone more than 2 (and preferably not more than 1.5) doublings from the time that the cells were collected or otherwise obtained until the genetic engineering of step c) is complete.

In a related aspect, the invention is directed to a method of producing a population of genetically engineered target cells from a sample comprising target cells that are not terminally differentiated by: a) obtaining a sample comprising target cells that are not terminally differentiated; b) separating the target cells from other cells, particles or unwanted materials to obtain an enriched population of target cells; and c) genetically engineering the target cells in the enriched population of cells with a nucleic acid (generally nucleotide) sequence to produce genetically engineered target cells with a therapeutically useful phenotype. A central feature of the method is that the proliferation and/or differentiation of target cells is minimized until within three days (and, in other embodiments within two days, one day, five hours, three hours or one hour) of the initiation of genetic engineering so as to maintain the cells in the same developmental state that they were in when first obtained.

Since cell division is needed for cells to integrate recombinant sequences, one or more factors that promote cell division or that otherwise re-direct differentiation may be added to target cells prior to genetic engineering. In order to minimize the overall number of divisions, such factors should not be added more than two or three days before genetic engineering is initiated. Alternatively the addition of factors may be delayed to one day (or alternatively, 5 hours, three hours or one hour) before genetic engineering is initiated. Such agents may also be given during or after the initiation of genetic engineering.

Factors promoting cell division or that otherwise re-direct differentiation of target cells that may be used include agents released by immune cells, cytokines, peptides, peptide-receptor complexes, antibodies either alone or in conjunction with other costimulatory molecules. Preferably cell division should be promoted while, as much as possible, maintaining target cells in an early developmental stage (e.g., as naïve T cells or T stem cells). When possible, the genetic engineering of cells should be initiated within 1 week after the sample comprising target cells is collected or otherwise obtained (and preferably, within 3 days, 1 day, or 6 hours). After genetic engineering, a further separation may be performed to remove reagents and stimulatory factors. This should typically be done within one or two days after genetic engineering is completed. Preferably, the reagents and any other stimulatory factors are removed using a size based separation procedure such as DLD. Although not preferred, after purification, cells can be stored, e.g., by freezing, for period of time prior to initiating genetic engineering.

As discussed above, samples may have contaminants in the form of cells, proteins or other factors that act as agents that promote the unwanted proliferation or differentiation. In blood samples or products derived from blood, contaminants would include platelets and/or one or more factors released by platelets. The separation should reduce such contaminants by at least 70% compared to the sample before separation and/or reduce the ratio of contaminants to target cells by at least 70% (and preferably 80%, 90% or 95%). In general, and particularly for samples such as blood, apheresis samples or leukapheresis samples, the ratio of platelets to target cells should be reduced as much and as quickly as possible. In this regard, it is preferred that the separation step be initiated within 5 hours (and more preferably 3 hours, two hours or one hour) after the sample comprising target cells is obtained.

The target cells may be any of those referred to above with the most preferred being T cells and particularly naive T cells or T stem cells. These may be collected or otherwise obtained in, inter alia, blood samples, apheresis samples or leukapheresis samples and a primary objective is to reduce the relative number of platelets in these sample as rapidly and thoroughly as possible using a method that maintains T cell viability and that minimizes the activation of the cells. When T cells are the target cells, antibodies or other factors may be added to the sample when obtained or during processing to block or reversibly inhibit the action of costimulators needed for the activation of the target T cells.

The separation of target cells from contaminants in step b) may be performed using any method, including size and/or affinity based separation methods, and without the addition of factors that promote the proliferation or differentiation of the target cells or that otherwise redirect the proliferation or differentiation of the cells. Also, it is preferred that centrifugation not be used as part of sample collection and processing or, if centrifugation was used during collection, e.g., as part of an apheresis or leukapheresis procedure, that it not be used thereafter, i.e., as part of the separation in step b) or as part of the genetic engineering in step c).

Preferably, using these methods, the amount of cells in samples not capable of entering into cell division should be reduced by at least 20% after the separation of step b) and the percentage of cells that effectively integrate nucleic acids and/or different forms of RNA, including miRNA and tRNA, should preferably be increased by at least 20%. In some embodiments, the number of cell divisions may be controlled entirely without the use of cell cycle or cell division inhibitors.

Preferably, at least 70% (and more preferably 80%, 90% or 95%) of the target cells should not have divided more than once from the time that they are collected or obtained until the separation of step b) is initiated. Similarly at least 70% (and preferably 80%, 90% or 95%) of the target cells should not have not been activated or divided more than once at the time that the genetic engineering of step c) is initiated, i.e., at the time that cells are first combined with the recombinant nucleic acids being transferred into the cells. Similarly, it is preferred that 70% (and preferably 80%, 90% or 95%) of the target cells not have divided more than twice (and more preferably not more than once) at the time that the genetic engineering of step c) is complete and/or that the population of cells not have undergone more than 2 (and preferably not more than 1.5) doublings from the time that the cells were collected or obtained until the genetic engineering of step c) is complete.

The most preferred methods involve the processing of T cells for therapeutic use. The T cells may be collected from a patient that will be treated with the therapeutic cells produced. Typically the sample will be whole blood, an apheresis or leukapheresis sample and, as discussed above, the target cells in the samples should be separated from platelets and other contaminants soon after collection. In addition, an agent that reversibly blocks the activation of T cells may be added to the sample before or during purification. Thus, antibodies or other factors may be added that block or reversibly inhibit the action of costimulators needed for the activation of the target T cells or that reversibly block the activation of T cells by inhibiting the binding of the T cell receptor to antigen and/or the signaling that results from antigen binding. In this way, the method may significantly increase the amount of cells capable of effectively being transformed with nucleic acids and/or different forms of RNA, including miRNA and tRNA. In an especially preferred embodiment, CAR T cells may be made by genetically engineering the T cells to produce chimeric antigen receptors (CARs) on their surface.

Using the above method, the yield of genetically engineered target cells having a desired phenotype should be at least at least 25% (and in some instances at least 50% or 75%) higher than the percentage in the unprocessed sample. By increasing the number of therapeutic cells that are at an early stage of development, e.g., that are stem cells, a preparation should be obtained that is therapeutically more effective.

In addition to the methods described above, the invention includes the cells produced by the methods, pharmaceutical compositions comprising these cells and methods of treating or preventing a disease or condition in a patient by administering a therapeutically effective amount of the pharmaceutical compositions. The most preferred cells are CAR T cells and the most preferred treatment is CAR T cell therapy.

### Definitions

Apheresis: As used herein this term refers to a procedure in which blood from a patient or donor is separated into its components, e.g., plasma, white blood cells and red blood cells. More specific terms are “plateletpheresis” (referring to the separation of platelets) and “leukapheresis” (referring to the separation of leukocytes). In this context, the term “separation” refers to the obtaining of a product that is enriched in a particular component compared to whole blood and does not mean that absolute purity has been attained.

CAR T cells: The term “CAR” is an acronym for “chimeric antigen receptor.” A “CAR T cell” is therefore a T cell that has been genetically engineered to express a chimeric receptor.

CAR T cell therapy: This term refers to any procedure in which a disease is treated with CAR T cells. Diseases that may be treated include hematological and solid tumor cancers, autoimmune diseases and infectious diseases.

Carrier: As used herein, the term “carrier” refers to an agent, e.g., a bead, or particle, made of either biological or synthetic material that is added to a preparation for the purpose of binding directly or indirectly (i.e., through one or more intermediate cells, particles or compounds) to some or all of the compounds or cells present. Carriers may be made from a variety of different materials, including DEAE-dextran, glass, polystyrene plastic, acrylamide, collagen, and alginate and will typically have a size of 1-1000 μm. They may be coated or uncoated and have surfaces that are modified to include affinity agents that recognize antigens or other molecules on the surface of cells. The carriers may also be magnetized and this may provide an additional means of purification to complement DLD.

Carriers that bind “in a way that promotes DLD separation”: This term, refers to carriers and methods of binding carriers that affect the way that a cell behaves during DLD. Specifically, “binding in a way that promotes DLD separation” means that: a) the binding must exhibit specificity for a particular target cell type; and b) must result in a complex that provides for an increase in size of the complex relative to the unbound cell. In this regard, there should generally be an increase of at least 2 μm (and alternatively at least 20, 50, 100, 200, 500 or 1000% when expressed as a percentage). In cases where therapeutic or other uses require that target cells, proteins or other particles be released from complexes to fulfill their intended use, then the term “in a way that promotes DLD separation” also requires that the complexes permit such release, for example by chemical or enzymatic cleavage, chemical dissolution, digestion, due to competition with other binders, or by physical shearing (e.g., using a pipette to create shear stress) and the freed target cells must maintain activity; e.g., therapeutic cells after release from a complex must still maintain the biological activities that make them therapeutically useful.

Target cells: As used herein “target cells” are the cells that various procedures described herein require or that the procedures are designed to purify, collect, engineer etc. What the specific cells are will depend on the context in which the term is used.

Isolate, purify: Unless otherwise indicated, these terms, as used herein, are synonymous and refer to the enrichment of a desired product relative to unwanted material. The terms do not necessarily mean that the product is completely isolated or completely pure. For example, if a starting sample had a target cell that constituted 2% of the cells in a sample, and a procedure was performed that resulted in a composition in which the target cell was 60% of the cells present, the procedure would have succeeded in isolating or purifying the target cell.

Bump Array: The terms “bump array” and “obstacle array” are used synonymously herein and describe an ordered array of obstacles that are disposed in a flow channel through which a cell or particle-bearing fluid can be passed.

Deterministic Lateral Displacement: As used herein, the term “Deterministic Lateral Displacement” or “DLD” refers to a process in which particles are deflected on a path through an array, deterministically, based on their size in relation to some of the array parameters. This process can be used to separate cells, which is generally the context in which it is discussed herein. However, it is important to recognize that DLD can also be used to concentrate cells and for buffer exchange.

Critical size: The “critical size” or “predetermined size” of particles passing through an obstacle array describes the size limit of particles that are able to follow the laminar flow of fluid. Particles larger than the critical size can be ‘bumped’ from the flow path of the fluid while particles having sizes lower than the critical size (or predetermined size) will not necessarily be so displaced.

Fluid flow: The terms “fluid flow” and “bulk fluid flow” as used herein in connection with DLD refer to the macroscopic movement of fluid in a general direction across an obstacle array. These terms do not take into account the temporary displacements of fluid streams for fluid to move around an obstacle in order for the fluid to continue to move in the general direction.

Tilt angle ε: In a bump array device, the tilt angle is the angle between the direction of bulk fluid flow and the direction defined by alignment of rows of sequential (in the direction of bulk fluid flow) obstacles in the array.

Array Direction: In a bump array device, the “array direction” is a direction defined by the alignment of rows of sequential obstacles in the array. A particle is “bumped” in a bump array if, upon passing through a gap and encountering a downstream obstacle, the particle's overall trajectory follows the array direction (i.e., travels at the tilt angle c relative to bulk fluid flow). A particle is not bumped if its overall trajectory follows the direction of bulk fluid flow under those circumstances.

### DETAILED DESCRIPTION OF THE INVENTION

The text below provides guidance regarding methods disclosed herein and information that may aid in the making and use of devices involved in carrying out those methods.

### I. The Processing of Sample to Remove Platelets and Other Factors

The methods described herein are characterized, in part, by the removal of platelets from blood samples, or samples derived from blood, soon after cells are first collected and by processing cells in a way that controls the number of cell divisions that they undergo. The most preferred purification method is by microfluidic separation. This not only rapidly removes small factors that may be detrimental to a high yield of therapeutic cells, including T memory stem cells and central memory cells, but may also be used to wash cells. It can also be used in the rapid removal of reagents and other factors that may be introduced in the processing of cells.

The methods disclosed herein, especially DLD, should preferably be capable of removing about 3.5 logs of virus in one pass as opposed to about 2 logs expected with most other approaches. Through the removal of platelets and other detrimental factors, 2-13× more central memory T cells (Tcm) cells should preferably be obtained. The ability to process cells within an hour of collection may limit degradation that might otherwise occur in this process, and may be done with a minimal dilution of the sample. Although DLD is preferred, other methods of separation that are applied very rapidly after cell collection and which rapidly separate desired cells from platelets and small detrimental factors may be employed.

In addition to eliminating detrimental factors, the invention may include the use of factors that direct cells to a therapeutically desirable phenotype. These may include: T cell activators; proteins (including affinity reagents, proteins, protein constructs, growth factors, specific antigens, engineered constructs); nucleic acids; nanomatrixes; micro-RNA; promoters; feedback inhibitors; and other agents that control division or promote integration of genetic content.

Separation Methods

The invention includes methods in which there is genetic engineering of a population of target cells. This is done by isolating the target cells from a crude fluid composition by performing a separation method, preferably a microfluidic method such as Deterministic Lateral Displacement (DLD) or an affinity based method.

An especially preferred separation method is DLD. In this type of separation, microfluidic devices are characterized by the presence of at least one channel which extends from a sample inlet to one or more fluid outlets, and which is bounded by a first wall and a second wall opposite from the first wall. An array of obstacles is arranged in rows in the channel, with each subsequent row of obstacles being shifted laterally with respect to a previous row. The obstacles are disposed in a manner such that, when a crude fluid composition is applied to an inlet of the device and passed through the channel, target cells flow to one or more collection outlets where an enriched product is collected, and contaminant cells or particles flow to one more waste outlets that are separate from the collection outlets.

Once the target cells have been purified using the device, they may be transfected or transduced with nucleic acids designed to impart upon the cells a desired phenotype, e.g., to express a chimeric molecule that makes the cells of therapeutic value. The population of cells may then be expanded by culturing in vitro.

In a preferred embodiment, the crude fluid composition is blood or, more preferably, a preparation of leukocytes that has been obtained by performing apheresis or leukapheresis on the blood of a patient. Preferred target cells include T cells, B-cells, NK-cells, monocytes and progenitor cells, with T cells being the most preferred. Apart from leukocytes, other types of cells, e.g., dendritic cells or stem cells, may also serve as target cells.

In general, crude fluid compositions containing target cells should be processed without freezing (at least up until the time that they are genetically engineered), and, preferably, at the site of collection. The crude fluid composition will preferably be the blood of a patient, and more preferably be a composition containing leukocytes obtained as the result of performing apheresis or leukapheresis on such blood. However, the term “crude fluid composition” also includes bodily fluids such as lymph or synovial fluid as well as fluid compositions prepared from bone marrow or other tissues. The crude fluid composition may also be derived from tumors or other abnormal tissue.

Although it is not essential that target cells be bound to a carrier before being genetically engineered, either before or after separation is first performed, they may be bound to one or more carriers provided that the carriers do not activate the cells. The exact means by which this occurs is not critical to the invention but binding should preferably be done “in a way that promotes DLD separation.” This term, as used in the present context, means that the method must ultimately result in binding that exhibits specificity for a particular target cell type, that provides for an increase in size of the complex relative to the unbound cell of at least 2 μm (and alternatively at least 20, 50, 100, 200, 500 or 1000% when expressed as a percentage) and, in cases where therapeutic or other uses require free target cells, that allow the target cell to be released from complexes by chemical or enzymatic cleavage, chemical dissolution, digestion, due to competition with other binders, by physical shearing, e.g., using a pipette to create shear stress, or by other means.

In one embodiment, the carriers have on their surface an affinity agent (e.g., an antibody) that allows the carriers to bind directly to the target cells with specificity. As used in this context, the word “specificity” means that at least 100 (and preferably at least 1000) target cells will be bound by carrier in the crude fluid composition relative to each non-target cell bound. In cases where the carrier binds after target cells in samples are separated, the binding may occur either before the target cells are genetically engineered or after.

Making of CAR T Cells

Methods for making and using CAR T cells are well known in the art. Procedures have been described in, for example, U.S. Pat. Nos. 9,629,877; 9,328,156; 8,906,682; US 2017/0224789; US 2017/0166866; US 2017/0137515; US 2016/0361360; US 2016/0081314;US 2015/0299317; and US 2015/0024482; each of which is incorporated by reference herein in its entirety.

In general, CAR T cells may be made by obtaining a crude fluid composition comprising T cells and performing DLD on the composition using a microfluidic device. Generally, the crude fluid composition comprising T cells will be an apheresis or leukapheresis product derived from the blood of a patient and containing leukocytes.

The microfluidic device should preferably have at least one channel extending from a sample inlet to one or more fluid outlets, wherein the channel is bounded by a first wall and a second wall opposite from the first wall. An array of obstacles is preferably arranged in rows in the channel, each subsequent row of obstacles being shifted laterally with respect to a previous row. These obstacles are disposed in a manner such that, when the crude fluid composition comprising T cells is applied to an inlet of the device and fluidically passed through the channel, the T cells flow to one or more collection outlets where an enriched product is collected and other cells (e.g., red blood cells, and platelets) or other particles of a different (generally smaller) size than the T cells flow to one more waste outlets that are separate from the collection outlets. Once obtained, the T cells are genetically engineered to produce chimeric antigen receptors (CARs) on their surface using procedures well established in the art. These receptors should generally bind antigens that are on the surface of a cell associated with a disease or abnormal condition. For example, the receptors may bind antigens that are unique to, or overexpressed on, the surface of cancer cells. In this regard, CD19 may sometimes be such an antigen.

Treating Cancer, Autoimmune Disease or Infectious Disease Using Cells In another aspect, the invention is directed to a method of treating a patient for a disease using cells prepared using the methods described herein. For example, CAR T cells may be used to treat an autoimmune disease, an infectious disease or cancer by administering the cells to a patient. Generally the patent treated should be the same patient that gave the blood from which the T cells were isolated.

### II. Designing Microfluidic Plates

Cells, particularly cells in compositions prepared by apheresis or leukapheresis, may be isolated using microfluidic devices. The preferred method is DLD using a device that contains a channel through which fluid flows from an inlet at one end of the device to outlets at the opposite end. Basic principles of size based microfluidic separations and the design of obstacle arrays for separating cells have been provided elsewhere (see, US 2014/0342375; US 2016/0139012; U.S. Pat. Nos. 7,318,902 and 7,150,812, which are hereby incorporated herein in their entirety) and are also summarized in the sections below.

During DLD, a fluid sample containing cells is introduced into a device at an inlet and is carried along with fluid flowing through the device to outlets. As cells in the sample traverse the device, they encounter posts or other obstacles that have been positioned in rows and that form gaps or pores through which the cells must pass. Each successive row of obstacles is displaced relative to the preceding row so as to form an array direction that differs from the direction of fluid flow in the flow channel. The “tilt angle” defined by these two directions, together with the width of gaps between obstacles, the shape of obstacles, and the orientation of obstacles forming gaps are primary factors in determining a “critical size” for an array. Cells having a size greater than the critical size travel in the array direction, rather than in the direction of bulk fluid flow and particles having a size less than the critical size travel in the direction of bulk fluid flow. In devices used for blood, apheresis or leukapheresis compositions, array characteristics may be chosen that result in white blood cells being diverted in the array direction whereas red blood cells and platelets continue in the direction of bulk fluid flow. In order to separate a chosen type of leukocyte from others having a similar size, a carrier may then be used that binds to that cell in a way that promotes DLD separation and which thereby results in a complex that is larger than uncomplexed leukocytes. It may then be possible to carry out a separation on a device having a critical size smaller than the complexes but bigger than the uncomplexed cells.

The obstacles used in devices may take the shape of columns or be triangular, square, rectangular, diamond shaped, trapezoidal, hexagonal or teardrop shaped. In addition, adjacent obstacles may have a geometry such that the portions of the obstacles defining the gap are either symmetrical or asymmetrical about the axis of the gap that extends in the direction of bulk fluid flow.

### III. Making and Operating Microfluidic Devices

General procedures for making and using microfluidic devices that are capable of separating cells on the basis of size are well known in the art. Such devices include those described in U.S. Pat. Nos. 5,837,115; 7,150,812; 6,685,841; 7,318,902; 7,472,794; and 7,735,652; all of which are hereby incorporated by reference in their entirety. Other references that provide guidance that may be helpful in the making and use of devices for the present invention include: U.S. Pat. Nos. 5,427,663; 7,276,170; 6,913,697; 7,988,840; 8,021,614; 8,282,799; 8,304,230; 8,579,117; US 2006/0134599; US 2007/0160503; US 20050282293; US 2006/0121624; US 2005/0266433; US 2007/0026381; US 2007/0026414; US 2007/0026417; US 2007/0026415; US 2007/0026413; US 2007/0099207; US 2007/0196820; US 2007/0059680; US 2007/0059718; US 2007/005916; US 2007/0059774; US 2007/0059781; US 2007/0059719; US 2006/0223178; US 2008/0124721; US 2008/0090239; US 2008/0113358; and WO2012094642 all of which are also incorporated by reference herein in their entirety. Of the various references describing the making and use of devices, U.S. Pat. No. 7,150,812 provides particularly good guidance and U.S. Pat. No. 7,735,652 is of particular interest with respect to microfluidic devices for separations performed on samples with cells found in blood (in this regard, see also US 2007/0160503).

A device can be made using any of the materials from which micro- and nano-scale fluid handling devices are typically fabricated, including silicon, glasses, plastics, and hybrid materials. A diverse range of thermoplastic materials suitable for microfluidic fabrication is available, offering a wide selection of mechanical and chemical properties that can be leveraged and further tailored for specific applications.

Techniques for making devices include Replica molding, Softlithography with PDMS, Thermoset polyester, Embossing, Injection Molding, Laser Ablation and combinations thereof. Further details can be found in “Disposable microfluidic devices: fabrication, function and application” by Fiorini, et al. (BioTechniques 38:429-446 (March 2005)), which is hereby incorporated by reference herein in its entirety. The book “Lab on a Chip Technology” edited by Keith E. Herold and Avraham Rasooly, Caister Academic Press Norfolk UK (2009) is another resource for methods of fabrication, and is hereby incorporated by reference herein in its entirety.

To reduce non-specific adsorption of cells or compounds, e.g., released by lysed cells or found in biological samples, onto the channel walls, one or more walls may be chemically modified to be non-adherent or repulsive. The walls may be coated with a thin film coating (e.g., a monolayer) of commercial non-stick reagents, such as those used to form hydrogels. Additional examples of chemical species that may be used to modify the channel walls include oligoethylene glycols, fluorinated polymers, organosilanes, thiols, poly-ethylene glycol, hyaluronic acid, bovine serum albumin, poly-vinyl alcohol, mucin, poly-HEMA, methacrylated PEG, and agarose. Charged polymers may also be employed to repel oppositely charged species. The type of chemical species used for repulsion and the method of attachment to the channel walls can depend on the nature of the species being repelled and the nature of the walls and the species being attached. Such surface modification techniques are well known in the art.

All references cited herein are fully incorporated by reference. Having now fully described the invention, it will be understood by one of skill in the art that the invention may be performed within a wide and equivalent range of conditions, parameters and the like, without affecting the spirit or scope of the invention or any embodiment thereof.

1-87. (canceled)

88. A method of producing a population of genetically engineered target cells from a sample comprising target cells that are not terminally differentiated, comprising:
a) obtaining a sample comprising said target cells together with contaminant cells, proteins or other factors that act as agents that promote the proliferation or differentiation of the target cells;
b) separating the target cells from the sample obtained in step a) using a size and/or affinity based separation method to obtain an enriched population of target cells wherein the contaminants are reduced by at least 70% compared to the sample before separation and/or the ratio of contaminants to target cells is at least 70% lower than in the sample originally obtained in step a);
c) genetically engineering the target cells in the enriched population of cells obtained in step b), with a nucleotide sequence to produce genetically engineered target cells with a therapeutically useful phenotype;
wherein, no factors that promote, or otherwise re-direct, the proliferation or differentiation of the target cells are added more than three days before the genetic engineering of step c) is initiated.

89. The method of claim 88, wherein the target cells are T cells and the “one or more factors that promote or otherwise re-direct the proliferation or differentiation of target cells” comprises cytokines, peptides, peptide receptor complexes, antibodies either alone or in conjunction with other costimulatory molecules.

90. The method of claim 88, wherein a factor that promotes the proliferation of target cells is added within three days prior to the time when genetic engineering is initiated and wherein the target cells are not centrifuged after sample is obtained.

91. The method of claim 88, wherein the target cells are selected from the group consisting of:
a) leukocytes, including neutrophils, basophils, eosinophils, lymphocytes (including B cells, T cells and natural killer cells); monocytes, macrophages, mast cells, dendritic cells;
b) stem cells including:
i) stem cells that develop into leukocytes such as stem cells with CD34 and/or CD38 markers and leukocyte lineage negative cells; and
ii) stem cells that develop into cells other than leukocytes;
c) erythroid precursor cells.

92. The method of claim 88, wherein the size based separation method is deterministic lateral displacement (DLD) on a microfluidic device.

93. The method of claim 88, wherein at least 80% of the target cells do not divide more than once from the time that they are obtained until the separation of paragraph b) is initiated.

94. The method of claim 88, wherein the sample is an apheresis or leukapheresis sample.

95. The method of claim 88, wherein, after step c), the genetically engineered cells are:
d) cultured to expand their number; and
e) transferred into a pharmaceutical composition for administration to a patient.

96. The method of claim 88, wherein the separation step in part b) is initiated within 5 hours after the sample comprising target cells is obtained.

97. The method of claim 88 wherein one or more agents are added to the sample of target cells before or during steps a) to c) to reversibly inhibit proliferation and/or differentiation.

98. A method of producing a population of genetically engineered target cells from a sample comprising target cells that are not terminally differentiated, comprising:
a) obtaining the sample comprising target cells that are not terminally differentiated;
b) separating the target cells from the sample obtained in step a) from other cells, particles or unwanted materials to obtain an enriched population of target cells;
c) genetically engineering the target cells in the enriched population of cells to produce genetically engineered target cells with a therapeutically useful phenotype;
wherein the proliferation and/or differentiation of target cells is minimized until initiation of genetic engineering of step c), so as to maintain the cells in the same developmental state that they were in when first obtained.

99. The method of claim 98, wherein one or more factors that promote the proliferation or differentiation of target cells are added at no more than three days before the genetic engineering of cells is initiated.

100. The method of claim 99, wherein the target cells are T cells and the “one or more factors that promote or otherwise re-direct the proliferation or differentiation of target cells” comprises cytokines, peptides, peptide-receptor complexes, antibodies either alone or in conjunction with other costimulatory molecules.

101. The method of claim 98, wherein the target cells are selected from the group consisting of:
a) leukocytes, including neutrophils, basophils, eosinophils, lymphocytes (including B cells, T cells and natural killer cells); monocytes, macrophages, mast cells, dendritic cells;
b) stem cells including:
i) stem cells that develop into Leukocytes such as stem cells with CD34 and/or CD38 markers and leukocyte lineage negative cells; and
ii) stem cells that develop into cells other than leukocytes;
c) erythroid precursor cells.

102. The method of claim 98, wherein the separation in step b) is performed using a size and/or affinity based separation method to obtain an enriched population of target cells and the target cells are not centrifuged after sample has been obtained.

103. The method of claim 98, wherein the target cells are T cells and antibodies or other factors are added to the sample or elsewhere during the process that block or reversibly inhibit the action of costimulators needed for the activation of the target T cells.

104. The method of claim 98, wherein an agent is added to the sample or during the processing of target cells that reversibly blocks the activation of T cells by inhibiting the binding of, and/or signaling from, the T cell receptor in response to antigen binding.

105. The method of claim 98, wherein the method reduces by at least 20% the amount of cells in the biological sample not capable of entering into cell division and increases the percentage of cells that effectively integrate nucleic acids and/or different forms of RNA, including miRNA and tRNA.

106. The method of claim 98, wherein at least 80% of the target cells have not been activated at the time that the separation of paragraph b) is completed.

107. A method of treating or preventing a disease or condition in a patient comprising administering to said patient a therapeutically effective amount of a pharmaceutical composition comprising cells made by the method of claim 98.
